(19) United States
(12) Patent Application Publication
(10) Pub. No.: US 2026/0041801 A1
(43) Pub. Date: Feb. 12, 2026

(54) Handcuff Disinfecting and Sanitizing Device

(71) Applicant: Robert Oaks, Spring, TX (US)

(72) Inventor: Robert Oaks, Spring, TX (US)

(21) Appl. No.: 19/200,917

(22) Filed: May 7, 2025

Related U.S. Application Data
(60) Provisional application No. 63/679,650, filed on Aug. 6, 2024.

(51) Int. Cl. A61L 2/18; A61L 2/24; A61L 101/34; E05B 75/00

(52) U.S. Cl. CPC A61L 2/183; A61L 2/24; A61L 2101/34; A61L 2202/11; A61L 2202/121; A61L 2202/122; A61L 2202/14; A61L 2202/16; E05B 75/005

(57) ABSTRACT

The present invention is a disinfecting and sanitizing device designed for disinfecting restraining devices such as handcuffs and leg shackles. The device comprises a cylindrical disinfectant liquid storage member, configured to store a disinfectant solution, and a circular lid that securely seals the storage member. The underside of the lid features a pair of integrated hooks, each with a U-shaped bend to hold restraining devices securely in place, preventing dislodgement during the disinfecting process. In one embodiment, the device includes a drainage slot for automatically draining the disinfectant solution when the quality thereof falls below a set threshold, and an ozone sterilization module to provide additional disinfection. The disinfectant solution preferably has 90% isopropyl alcohol, and cleans the restraining devices by removing dirt, bacteria, and bodily fluids.

### CROSS-REFERENCE TO RELATED APPLICATION

The present application claims priority to, and the benefit of, U.S. Provisional Application No. 63/679,650, which was filed on Aug. 6, 2024, and is incorporated herein by reference in its entirety.

### FIELD OF THE INVENTION

The present invention generally relates to devices used for disinfecting and sanitizing restraining devices. More specifically, the invention pertains to a handcuffs disinfecting and sanitizing device designed to efficiently clean handcuffs, leg shackles, and similar restraining devices commonly used in law enforcement, military, and security applications. The device integrates a disinfectant liquid storage member with a sealing lid and incorporates hooks on the underside of the lid to securely hold a restraining device while the restraining device is immersed in a disinfectant solution. The device offers a portable, user-friendly solution for maintaining hygiene and preventing the spread of infectious materials, particularly in high-risk environments such as police departments and correctional facilities. Accordingly, the present disclosure makes specific reference thereto. Nonetheless, it is to be appreciated that aspects of the present invention are also equally applicable to other like applications, devices, and methods of manufacture.

### BACKGROUND

By way of background, restraining devices such as handcuffs are commonly used in law enforcement, correctional facilities, and military applications to restrain individuals such as suspects or prisoners. However, over time, the restraining devices can accumulate dirt, grime, bodily fluids, and contaminants such as saliva, blood, and bacteria. The contamination poses a hygiene concern, as handcuffs are frequently applied to multiple individuals throughout their use, often without any cleaning or disinfecting between applications. As a result, harmful pathogens can be transferred from one person to another, potentially spreading infections and diseases.

The failure to properly clean and sanitize handcuffs between uses increases the likelihood of cross-contamination, particularly in high-risk environments where bodily fluids may be present. Without a dedicated method to disinfect these restraints, officers and personnel risk exposure to germs, viruses, and other harmful microorganisms, which can lead to health issues for both the person wearing the handcuffs and the officer or personnel applying them. Officers generally have to manually wash handcuffs with soap and water after every use, which is not always possible but is also ineffective, laborious, and time-consuming. Individuals desire a more efficient, reliable, and hygienic solution to disinfecting and sanitizing handcuffs in order to prevent the spread of germs, bacteria, and bodily fluids.

Therefore, there exists a long-felt need in the art for a device that disinfects and sanitizes handcuffs, leg shackles, and similar restraining devices used in law enforcement, military, and security applications. There is a long-felt need for a portable and easy-to-use device that sanitizes handcuffs and other restraining devices between uses, preventing the transfer of germs, bacteria, bodily fluids, and other harmful pathogens. Additionally, there is a long-felt need for a solution that eliminates the time-consuming and often neglected task of manually washing handcuffs with soap and water. Furthermore, there exists a long-felt need for a cleaning system that prevents bodily fluids, germs, bacteria, etc., from being spread to different individuals and the arresting officer by keeping handcuffs clean. More specifically, there is a long-felt need for a disinfecting device that can accommodate various restraint sizes and is capable of providing thorough cleaning within a short time frame. Finally, there is a long-felt need for a device that enables police officers, military police, and others who use handcuffs to keep them clean and sanitary for new applications to suspects and prisoners.

The subject matter disclosed and claimed herein, in one embodiment, comprises a handcuffs disinfecting and sanitizing device that provides an efficient solution to disinfect handcuffs, leg shackles, and other similar restraining devices. The device includes a cylindrical disinfection liquid storage member that holds a disinfectant solution, such as 90% isopropyl alcohol. The storage member is sealed by a circular lid, which features integrated hooks that securely hold a restraining device in place when the restraining device is immersed in the disinfectant solution. In some embodiments, the device also includes a sealing gasket to prevent leakage. The device is portable and easy to use, providing a convenient solution for maintaining hygiene and preventing the spread of germs and bacteria between suspects, prisoners, and law enforcement officers.

In this manner, the handcuffs disinfecting and sanitizing device of the present invention fulfills the long-felt need for a quick, effective, and hygienic way to clean restraining devices in environments where cross-contamination is a concern. The device is designed to accommodate or hang (i.e., suspend) different types and sizes of restraints, offering versatility in various settings. Further, the device enables police officers, military police, and others who use handcuffs to keep them clean and sanitary for new applications to suspects and prisoners.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a handcuffs disinfecting and sanitizing device. The handcuffs disinfecting and sanitizing device comprises a disinfectant liquid storage member having a substantially cylindrical shape, wherein the disinfectant liquid storage member is configured to store a disinfectant solution. A circular lid has a periphery adapted to securely seal the disinfection liquid storage member, wherein the bottom surface of the lid includes a pair of integrated hooks extending downward, each hook has a U-shaped bend at the respective bottom end. The U-shaped bend of each hook is configured to securely hold a restraining device, such as handcuffs, preventing dislodgement or falling off of the restraining device when immersed in the disinfectant solution.

In another aspect, each hook of the pair of hooks has a length of about 30% to 60% of the height of the disinfectant liquid storage member, and wherein the U-shaped bend of each hook is positioned to prevent dislodgement of a restraining device suspended on the hooks.

In one embodiment, a sealing gasket is disposed around the periphery of the circular lid, wherein the gasket automatically engages when the lid is pressed to seal the disinfectant liquid storage member and prevent leakage of the disinfectant solution.

In yet another aspect, a handcuffs disinfecting and sanitizing device is disclosed. The handcuffs disinfecting and sanitizing device includes a disinfectant liquid storage member configured to store a disinfectant solution, the disinfectant liquid storage member being substantially cylindrical with a diameter between about 8 inches and about 14 inches. A circular lid provides a sealed closure to the disinfectant liquid storage member, wherein the bottom surface of the lid includes a pair of integrated hooks extending downward, each hook has a U-shaped bend at its respective bottom end to secure a restraining device. A drainage slot is disposed at the base of the disinfectant liquid storage member, wherein the drainage slot is configured to automatically activate and drain the disinfectant solution when the solution level falls below a predetermined threshold quality level.

In another aspect, the handcuffs disinfecting and sanitizing device further includes an ozone sterilization module disposed within the disinfectant liquid storage member, wherein the module is configured to activate automatically upon detecting a restraining device, providing additional disinfection by generating ozone to kill pathogens not removed by the disinfectant solution.

In yet another aspect, the disinfectant solution comprises at least 90% isopropyl alcohol, and wherein the disinfectant liquid storage member is configured to clean a restraining device for a predetermined time period ranging from about 5 minutes to about 20 minutes.

In a further embodiment, a restraining device is placed onto the pair of hooks extending from the underside of a circular lid, wherein each hook has a U-shaped bend at the bottom end for securely holding the restraining device in place. The circular lid is secured onto the disinfectant liquid storage member to form a sealed closure, thereby preventing leakage of the disinfectant solution. The restraining device is immersed in the disinfectant solution stored within the disinfection liquid storage member, wherein the disinfectant solution cleans the restraining device by removing dirt, grime, bacteria, bodily fluids, and other debris.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of the handcuffs disinfecting and sanitizing device of the present invention in an open state in accordance with the disclosed structure;

FIG. 2 illustrates a perspective view of handcuffs being placed on the hooks of the handcuffs disinfecting and sanitizing device in accordance with the disclosed structure;

FIG. 3 illustrates a perspective view of the handcuffs being disinfected using the handcuffs disinfecting and sanitizing device of the present invention in accordance with the disclosed structure;

FIG. 4 illustrates a perspective view showing different sizes of the handcuffs disinfecting and sanitizing device of the present invention in accordance with the disclosed structure; and

FIG. 5 illustrates another embodiment of the handcuffs disinfecting and sanitizing device of the present invention in accordance with the disclosed structure.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a device that disinfects and sanitizes handcuffs, leg shackles, and similar restraining devices used in law enforcement, military, and security applications. There is a long-felt need for a portable and easy-to-use device that sanitizes handcuffs and other restraining devices between uses, preventing the transfer of germs, bacteria, bodily fluids, and other harmful pathogens. Additionally, there is a long-felt need for a solution that eliminates the time-consuming and often neglected task of manually washing handcuffs with soap and water. Furthermore, there exists a long-felt need for a cleaning system that prevents bodily fluids, germs, bacteria, etc., from being spread to different individuals and the arresting officer by keeping handcuffs clean. More specifically, there is a long-felt need for a disinfecting device that can accommodate various restraint sizes and is capable of providing thorough cleaning within a short time frame. Finally, there is a long-felt need for a device that enables police officers, military police, and others who use handcuffs to keep them clean and sanitary for new applications to suspects and prisoners.

The present invention, in one exemplary embodiment, is a handcuffs disinfecting and sanitizing device. The handcuffs disinfecting and sanitizing device includes a disinfectant liquid storage member configured to store a disinfectant solution, the disinfectant liquid storage member being substantially cylindrical with a diameter between about 8 inches and about 14 inches. A circular lid provides a sealed closure to the disinfectant liquid storage member, wherein the bottom surface of the lid includes a pair of integrated hooks extending downward, each hook has a U-shaped bend at its respective bottom end to secure a restraining device. A drainage slot is disposed at the base of the disinfectant liquid storage member, wherein the drainage slot is configured to automatically activate and drain the disinfectant solution when the solution level falls below a predetermined threshold quality level.

Reference will now be made in detail to the present preferred embodiments of the invention, examples of which are illustrated in the accompanying drawings. Wherever possible, the same reference numerals are used in the drawings and the description to refer to the same or like parts.

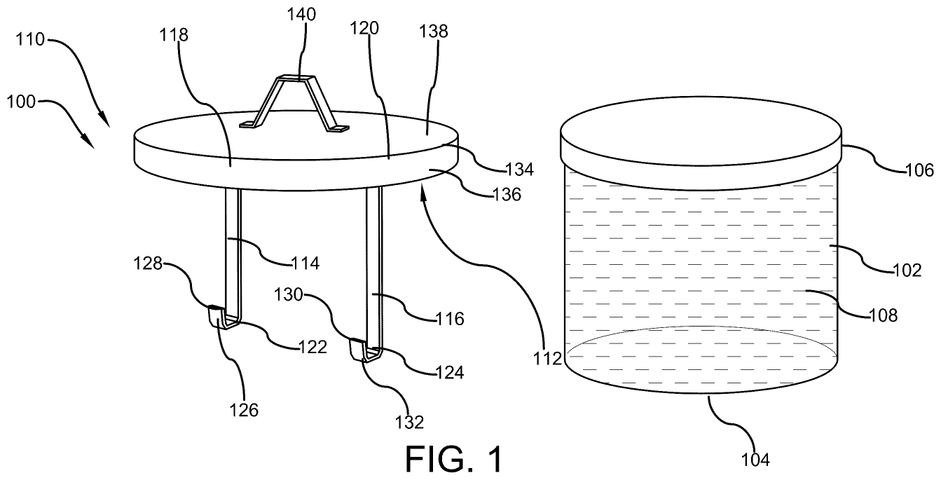

Referring initially to the drawings, FIG. 1 illustrates a perspective view of one potential embodiment of a restraining device (i.e., handcuffs) disinfecting and sanitizing device of the present invention in an open state in accordance with the disclosed structure. The restraining device or handcuffs disinfecting and sanitizing device 100 of the present invention is a specialized device designed for disinfecting and sanitizing handcuffs, leg shackles, and similar restraining devices used by law enforcement, military police, and security personnel. More specifically, the restraining device or handcuffs disinfecting and sanitizing device 100 prevents the spread of germs, bodily fluids, and other potentially harmful materials between suspects, prisoners, and officers.

The restraining device or handcuffs disinfecting and sanitizing device 100 is in the form of a portable and handheld device and includes a disinfectant liquid storage member 102. The disinfectant liquid storage member 102 is preferably made of plastic or glass and is durable, non-reactive to the disinfectant solution, and easy to clean. The disinfectant liquid storage member 102 is substantially cylindrical and includes a diameter from about 8 inches to about 14 inches, and a height from between 8 inches and 14 inches extending between the bottom end 104 and the top end 106 thereof. The disinfectant liquid storage member 102 is configured to store a disinfectant solution 108, preferably made up of at least 90% isopropyl alcohol.

A circular lid 110 made of metal or plastic is configured to provide a sturdy and sealed closure to the disinfectant liquid storage member 102. The circular lid 110 is adapted to the circumference and shape of the disinfectant liquid storage member 102 and provides an airtight and liquid-tight closure that prevents leakage of the disinfectant solution during the cleaning process. The bottom surface 112 of the lid 110 has a pair of integrated hooks 114, 116 extending on the underside of the lid 110. The hooks 114, 116 are identical, and each hook has a length of about 30%-60% of the height of the disinfectant liquid storage member 102, wherein the length of the hooks 114, 116 extend from the respective top ends 118, 120 to the respective bottom ends 122, 124.

Each hook of the pair of the hooks 114, 116 has a U-shaped bend disposed at the respective bottom end for enabling a secure fit to accommodate or hang (i.e., suspend) a restraining device. The free end 128 of the U-shaped bend 126 is positioned vertically above the bottom end 122 of the hook 114, and similarly, the free end 130 of the U-shaped bend 132 is positioned vertically above the bottom end 124 of the hook 116. The hooks 114, 116 enable that the handcuffs or similar restraints remain suspended and immersed securely in the disinfectant solution 108 without risk of falling off or being dislodged.

A sealing gasket 134 is disposed around the periphery 136 of the lid 110, and the gasket 134 engages automatically when the lid 110 is pressed, thereby sealing the disinfectant liquid storage member 102. The top surface 138 of the lid 110 has an ergonomic handle 140 disposed thereon. The handle 140 enables users to pull and push the lid 110 for securing the lid 110 to the disinfectant liquid storage member 102.

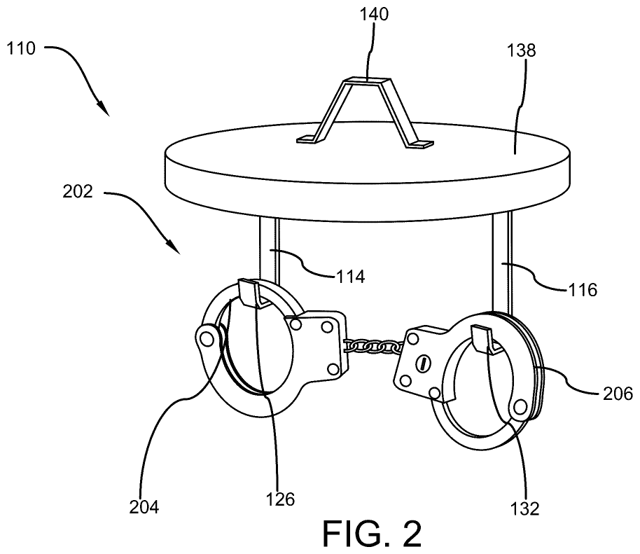

FIG. 2 illustrates a perspective view of handcuffs being placed on the hooks of the handcuffs disinfecting and sanitizing device in accordance with the disclosed structure. As illustrated, the handcuffs 202 are secured to the hooks 114, 116 and more specifically, a first strand 204 of the handcuffs 202 are hung on the first U-shaped bend 126 and a second strand 206 of the handcuffs 202 are hung on the second U-shaped bend 132, such that the handcuffs 202 are securely hung using the hooks 114, 116. The U-shaped bends 126, 132 eliminate the risk of falling off or dislodging of the handcuffs 202.

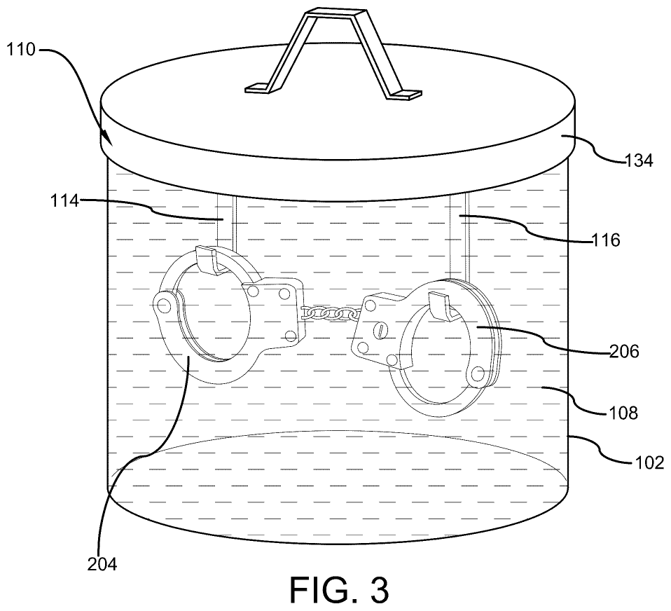

FIG. 3 illustrates a perspective view of the handcuffs being disinfected using the handcuffs disinfecting and sanitizing device of the present invention in accordance with the disclosed structure. For disinfection of a restraining device such as the handcuffs, the handcuffs 202 are secured to the hooks 114, 116 as described and illustrated in FIG. 2. When the handcuffs 202 are secured, the lid 110 is secured onto the disinfectant liquid storage member 102 for sealing the disinfectant liquid storage member 102. When lid 110 is secured, the handcuffs 202 are lowered into the disinfectant solution 108 stored in the disinfectant liquid storage member 102. The handcuffs 202 can be disinfected for a predetermined time period such as ranging from about 5 minutes to about 20 minutes. The disinfectant solution 108 cleans the handcuffs 202, removes dirt, grime, bacteria, bodily fluids, and other debris from the surface of the handcuffs 202.

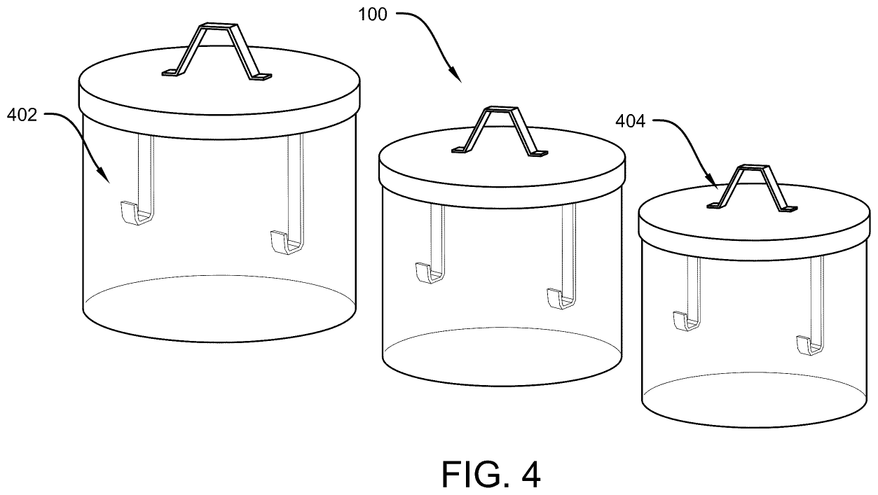

FIG. 4 illustrates a perspective view showing different sizes of the handcuffs disinfecting and sanitizing device of the present invention in accordance with the disclosed structure. In the present embodiment, the handcuffs disinfecting and sanitizing device 100 can be designed to accommodate or hang (i.e., suspend) different types of restraining devices. Some embodiments of the handcuffs disinfecting and sanitizing device 100 can be designed in larger sizes, such as the device 402, to cater to larger handcuffs, leg shackles, or other forms of restraint devices used by law enforcement or military personnel. For smaller restraining devices, a smaller device 404 can be used for disinfection and sanitization.

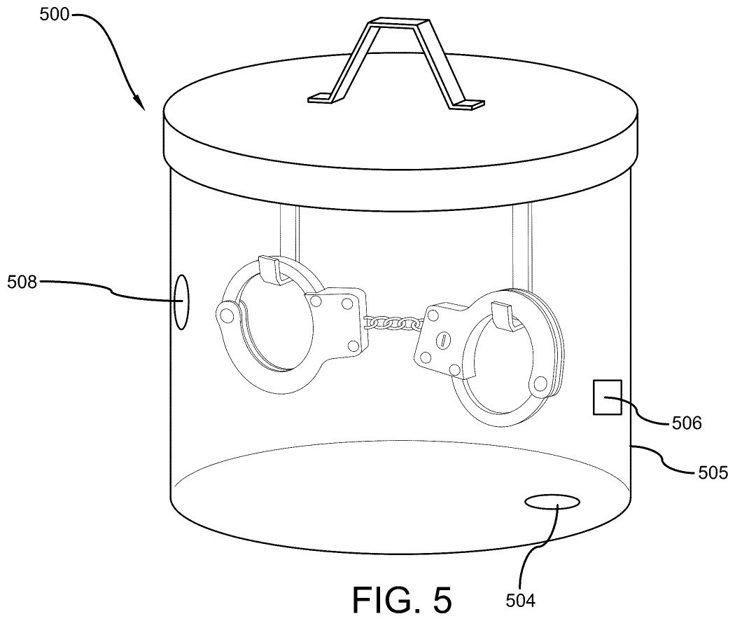

FIG. 5 illustrates another embodiment of the handcuffs disinfecting and sanitizing device of the present invention in accordance with the disclosed structure. In the present embodiment, the handcuffs disinfecting and sanitizing device 500 has a drainage slot or plug 502 disposed in the base 504 for draining the disinfectant solution. Further, the storage member 505 has an ozone sterilization module 506 for providing additional disinfection by killing pathogens that are harder to remove through liquid solutions alone. The ozone sterilization module 506 is configured to automatically detect a restraining device inside the handcuffs disinfecting and sanitizing device 500 for activation. Further, the drainage slot or plug 502 can be automatically activated when the disinfectant solution stored in the handcuffs disinfecting and sanitizing device 500 is below a threshold quality level. A sensor 508 disposed in the handcuffs disinfecting and sanitizing device 500 is configured to detect the level of contamination on the restraining device and adjusts the duration of the disinfecting cycle provided by the handcuffs disinfecting and sanitizing device 500.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein, “handcuffs disinfecting and sanitizing device”, “restraining device disinfecting and sanitizing device”, “portable handcuffs disinfecting and sanitizing system”, and “device” are interchangeable and refer to the restraining device disinfecting and sanitizing device 100, 500 of the present invention.

Notwithstanding the foregoing, the restraining device disinfecting and sanitizing device 100, 500 of the present invention can be of any suitable configuration as is known in the art without affecting the overall concept of the invention, provided that it accomplishes the above-stated objectives. One of ordinary skill in the art will appreciate that the restraining device disinfecting and sanitizing device 100, 500 as shown in the FIGS. are for illustrative purposes only, and that many other configurations of the restraining device disinfecting and sanitizing device 100, 500 are well within the scope of the present disclosure. Although the dimensions of the restraining device disinfecting and sanitizing device 100, 500 are important design parameters for user convenience, the restraining device disinfecting and sanitizing device 100, 500 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A disinfecting and sanitizing system comprising:
a disinfectant solution storage member;
a lid;
a disinfectant solution; and
a restraining device;
wherein said disinfectant solution storage member is handheld;
wherein said disinfectant solution storage member having a material selected from the group consisting of a plastic and a glass;
wherein said disinfectant solution storage member is cylindrical including a diameter from 8 inches to 14 inches;
wherein said lid is circular and selectively removable from said disinfectant solution storage member;
wherein said disinfectant solution storage member selectively sealable with said lid;
wherein said lid having a plurality of hooks mounted to an underside of said lid and extending therefrom for attaching said restraining device;
wherein suspending said restraining device from said underside of said lid; and
further wherein immersing said restraining device in said disinfectant solution within said disinfectant solution storage member for disinfecting said restraining device.

2. The disinfecting and sanitizing system of claim 1, wherein said disinfectant solution comprising rubbing alcohol.

3. The disinfecting and sanitizing system of claim 2, wherein said rubbing alcohol is at least 90% isopropyl alcohol.

4. The disinfecting and sanitizing system of claim 3, wherein said restraining device is a pair of handcuffs.

5. The disinfecting and sanitizing system of claim 4, wherein said disinfectant solution storage member is portable.

6. The disinfecting and sanitizing system of claim 5, wherein said plurality of hooks is a pair of hooks, and further wherein each of said pair of hooks having a length from 30% to 60% of a height of said disinfectant solution storage member.

7. The disinfecting and sanitizing system of claim 6, wherein each of said pair of hooks having a U-shaped bend at a terminal bottom end for the attaching and the suspending of said restraining device.

8. The disinfecting and sanitizing system of claim 7, wherein said lid having a sealing gasket disposed around a periphery of said lid for sealing said disinfectant solution storage member.

9. A disinfecting and sanitizing system comprising:
a disinfectant solution storage member;
a lid;
a disinfectant solution; and
a restraining device;
wherein said disinfectant solution storage member is handheld;
wherein said disinfectant solution storage member having a material selected from the group consisting of a plastic and a glass;
wherein said disinfectant solution storage member is cylindrical including a diameter from 8 inches to 14 inches;
wherein said lid is circular and selectively removable from said disinfectant solution storage member;
wherein said disinfectant solution storage member selectively sealable with said lid;
wherein said lid having a plurality of hooks mounted to an underside of said lid and extending therefrom for attaching said restraining device;
wherein suspending said restraining device from said underside of said lid;
wherein immersing said restraining device in said disinfectant solution within said disinfectant solution storage member;
wherein said top surface of said lid having a handle for grasping said lid to push and pull said lid for selectively securing said lid to said disinfectant solution storage member;
wherein said disinfectant solution comprising rubbing alcohol;
wherein said rubbing alcohol is at least 90% isopropyl alcohol; and
further wherein said restraining device is a pair of handcuffs.

10. The disinfecting and sanitizing system of claim 9, wherein said disinfectant solution storage member is portable.

11. The disinfecting and sanitizing system of claim 10, wherein said plurality of hooks is a pair of hooks, and further wherein each of said pair of hooks having a length from 30% to 60% of a height of said disinfectant solution storage member.

12. The disinfecting and sanitizing system of claim 11, wherein each of said pair of hooks having a U-shaped bend at a terminal bottom end for the attaching and the suspending of said restraining device.

13. The disinfecting and sanitizing system of claim 12, wherein said lid having a sealing gasket disposed around a periphery of said lid for sealing said disinfectant solution storage member.

14. A method of disinfecting and sanitizing a restraining device, the method comprising the steps of:
providing a disinfectant solution storage member, a lid, a disinfectant solution, and a restraining device, wherein said disinfectant solution storage member having a material selected from the group consisting of a plastic and a glass, wherein said disinfectant solution storage member is cylindrical including a diameter from 8 inches to 14 inches, further wherein said lid having a plurality of hooks mounted to an underside of said lid and extending therefrom and a handle mounted to a top surface of said lid for grasping said lid to push and pull said lid;
removing said lid from said disinfectant solution storage member;
attaching said restraining device to said plurality of hooks;
securing said lid to said disinfectant solution storage member;
suspending said restraining device from said underside of said lid; and
immersing said restraining device in said disinfectant solution within said disinfectant solution storage member, wherein said disinfectant solution comprising isopropyl alcohol and further wherein said restraining device is a pair of handcuffs.

15. The method of disinfecting and sanitizing a restraining device of claim 14, wherein said disinfectant solution storage member is portable.

16. The method of disinfecting and sanitizing a restraining device of claim 15, wherein said plurality of hooks is a pair of hooks, and further wherein each of said pair of hooks having a length from 30% to 60% of a height of said disinfectant solution storage member.

17. The method of disinfecting and sanitizing a restraining device of claim 16, wherein said disinfectant solution storage member is handheld.

18. The method of disinfecting and sanitizing a restraining device of claim 17, wherein said disinfectant solution storage member selectively sealable with said lid.

19. The method of disinfecting and sanitizing a restraining device of claim 18, wherein each of said pair of hooks having a U-shaped bend at a terminal bottom end for the attaching and the suspending of said restraining device.

20. The method of disinfecting and sanitizing a restraining device of claim 19, wherein said lid having a sealing gasket disposed around a periphery of said lid for sealing said disinfectant solution storage member.
